(19) United States
(12) Patent Application Publication
(10) Pub. No.: US 2009/0317844 A1
(43) Pub. Date: Dec. 24, 2009

(54) DIAGNOSTIC TEST FOR HEPATOCELLULAR CARCINOMA

(75) Inventor: Mochtar RIADY, Tangerang (ID)

Correspondence Address: BROWDY AND NEIMARK, P.L.L.C.;624 NINTH STREET, NW, SUITE 300, WASHINGTON, DC, 20001-5303, US

(73) Assignee: MOCHTAR RIADY INSTITUTE of NANOTECHNOLOGY, Tangerang (ID)

(21) Appl. No.: 12/144,958

(22) Filed: Jun. 24, 2008

(51) Int. Cl. G01N 33/53; C12M 1/34

(52) U.S. Cl. 435/7.93; 435/7.1; 435/287.1

(57) ABSTRACT

A diagnostic test for hepatocellular carcinoma is based on increases in the concentration of two biochemical markers in a biological sample, alpha-1-acid glycoprotein (AAG) or isoforms thereof and alpha-fetoprotein (AFP) or glycoforms thereof. Levels of these markers may be performed using an immunoassay. Also disclosed is a diagnostic kit for use in these assays that comprises reagents for detecting and/or measuring in AAG and AFP in blood, serum or tissue samples. A point-of-care device for the performance of such measurements is also disclosed.

### BACKGROUND OF THE INVENTION

1. Field of the Invention

The invention relates generally to methods, tests, and kits to detect liver damage in mammals. In particular, the present invention relates to a diagnostic test for hepatocellular carcinoma utilizing two biochemical markers present in a biological sample such as blood serum.

2. Description of the Background Art

The prognosis and survival of patients with hepatocellular carcinoma (HCC) is heavily affected by the disease stage at the time of diagnosis. Initial diagnosis may be difficult as imaging modalities, such as ultrasonography, are currently limited by their low positive predictive values (Lopez, 205, Clin Biochem Rev 26:65-79; Maringhini et. al., 1984, Cancer 54:2924-26). Imaging of HCC is complicated because the tumor has a varied radiologic appearance and frequently coexists with regenerative and dysplastic nodules in the cirrhotic liver. Magnetic resonance imaging (MRI) is also described as insensitive for the detection of HCC nodules<2 cm (Lopez, supra). Positron emission tomography utilizing the most common tracer fluorodeoxyglucose also shows poor sensitivity in diagnosing HCC (He & Guo, 2008, Postgrad Med J. 84:246-51).

It has been widely reported that alpha-fetoprotein (AFP), the only serological marker currently available in clinical practice, is not a sufficiently reliable marker to identity HCC patients, mainly because of its poor sensitivity (Taketa, 1990, Hepatology 12:1420-32; Sherman & Klein, 2004, Hepatology 40:1465-73; Marrero & Lok, 2004, Gastroenterology 127:S113-9).

Several studies indicate that high levels of AFP are related to poor prognosis as well as the histologic grade of malignancy. Those with high serum AFP levels at the time of HCC diagnosis have more unfavorable outcomes compared to patients with low AFP levels (Johnson et al., 1981, Br J Cancer 44:502-5; Shirabe et al., 1997, J Surg Oncol 64:143-6). Moreover, equivocal AFP concentrations are common in non-malignant chronic liver diseases (CLD), and the sensitivity of the AFP test for HCC in this setting thus tends to be low (Giannelli et al., 2007, Clinica Chimica Acta 383: 147-52). This observation leads to a critical need to review of how AFP can be employed to improve its utility in detection of HCC.

The availability of reliable markers for HCC would greatly improve the chances of detecting early stage HCC. Recent studies have demonstrated that serum alpha-1-acid glycoprotein (AAG) concentration is increased in patients with HCC and CLD, suggesting AAG may be a potential marker for cirrhosis and HCC (Song et al., 2003, Hepatol. Res. 26:311-7; Paul et al., 2006, Biomed Chromatog 20:1351-8; Kim et al., 2006, Clin. Chim. Acta, 369:46-51).

Recently, a combination test using AFP with serum haptoglobin (HP) and its glycoforms was used to improve diagnosis of HCC (Ang et al., 2006, J. Proteome Res. 5:2691-2700). However, while this test was able to improve the diagnosis of HCC it was not able to provide sufficient sensitivity, specificity and accuracy to enable this test replace other clinical tests currently in use. Accordingly, there is a continuing need for an accurate diagnostic test for HCC.

### SUMMARY OF THE INVENTION

The present inventor discovered that the combination of alpha-1-acid glycoprotein (AAG) and alpha-fetoprotein (AFP) shows high sensitivity and improves the accuracy of HCC diagnosis.

Accordingly, in a first aspect the present invention provides a method of detecting hepatocellular carcinoma in a mammal, said method comprising:

(i) obtaining a biological sample from a mammal suspected of hepatocellular carcinoma;

(ii) detecting the presence of AAG or isoforms thereof and AFP or glycoforms thereof and measuring the concentration of AAG and AFP present in said sample to obtain a quantified amount of each;

(iii) comparing the quantified amount of said AAG and AFP in step (ii) with a control sample, which control is a quantified amount of AAG and AFP derived from biological samples from mammals not suffering from hepatocellular carcinoma; and

(iv) determining the difference between the quantified amounts in step (ii) and step (iii);

wherein an increased amount of AAG and AFP in the sample in step (i) relative to the amount of AAG and AFP in the control is indicative of the presence of HCC.

In some embodiments, the quantified amount of AAG and AFP derived from biological samples from mammals not suffering from hepatocellular carcinoma are <680 μg/mL for AAG and <38 ng/ml for AFP.

Accordingly, in a second aspect the present invention provides a method of detecting hepatocellular carcinoma in a mammal, said method comprising:

(i) obtaining a biological sample from a mammal suspected of hepatocellular carcinoma;

(ii) detecting the presence of AAG or isoforms thereof and AFP or glycoforms thereof;

(iii) measuring the concentration of AAG and AFP present in said sample to obtain a quantified amount of each;

(iv) determining if said quantified amount of each are greater than 680 μg/mL for AAG and 38 ng/ml for AFP;

wherein the presence of HCC is indicated.

All mammals are capable of developing HCC. Accordingly, the methods disclosed herein are capable of use in the diagnosis of HCC in any mammal. In some embodiments the subject is a human or a mammal of economical importance and/or social importance to humans, for instance, carnivores other than humans (such as cats and dogs), swine (pigs, hogs, and wild boars), ruminants (such as cattle, oxen, sheep, giraffes, deer, goats, bison, and camels) and horses. The term does not denote a particular age. Thus, both adult and newborn subjects are intended to be covered.

Biological samples useful for practicing the methods of the invention include, but are not limited to, a tissue, or fluid such as bone marrow, plasma, serum, spinal fluid, lymph fluid, the sections of the respiratory, intestinal, or genitourinary tracts, tears, saliva, milk, whole blood, tumors and organs. In some embodiments the sample is blood.

Body fluids, which are preferred biological samples, may be conveniently obtained from a mammal by any conventional method known to those skilled in the art. For example, the biological sample may be obtained directly from the subject by a needle or swab or other suitable collection device.

Accordingly, in a third aspect the present invention provides a diagnostic kit for detecting hepatocellular carcinoma in a mammal, said kit comprising reagents for measuring the amount of AAG or isoforms thereof and AFP or glycoforms thereof present in a biological sample to derive a quantified amount of AAG and AFP.

In some embodiments, the reagents for measuring the amount of AAG and AFP include antibodies capable of selectively binding AAG and AFP. In some embodiment, the kits of the invention further comprise positive or negative controls including, for example, known quantities or amounts of AAG and AFP (e.g., 680 μg/mL for AAG and 38 ng/ml for AFP) or samples from mammals not suffering from hepatocellular carcinoma.

Accordingly, in a fourth aspect the present invention provides one or more antibodies capable of selectively binding to AAG and AFP.

In a fifth aspect the present invention provides a competitive enzyme linked immunosorbent assay (ELISA) kit for determining the HCC status of a mammalian subject, comprising a first antibody specific to AAG and a second antibody specific to AFP, wherein said first and second antibody can detect the presence of AAG and AFP in a biological sample taken from said subject.

In some embodiments, the kit of the present invention is embodied in a point of care device. Accordingly, a fifth aspect the present invention provides a device for determining the presence of AAG and AFP in a biological sample, comprising: a layer including a continuous porous medium deposited thereon; a fluid pathway defined by fluid barriers in the porous medium, at least a portion of the barriers defining a capillary channel, which fluid pathway comprises a constriction region immediately upstream of the capillary channel for constricting and directing fluid flow to the capillary channel; an analysis zone within the fluid pathway comprising a plurality of binding zones for binding and immobilizing the AAG and AFP, each binding zone being defined by a concentration of a binding reagent immobilized to the medium; and an application zone at the first end of the fluid pathway and upstream of the analysis zone, for receiving a biological sample suspected of containing AAG and AFP,

### BRIEF DESCRIPTION OF THE DRAWINGS

FIG. 1 shows a chromatogram profile of chronic liver (A) and HCC patient with low AFP concentrations (≦200 ng/mL) (B) and HCC patients with high AFP concentrations (>200 ng/mL) (C) using Agilent MRP-C18 at 280 nm. AAG (grey area) from HCC profile show higher concentration than chronic liver.

FIG. 2 shows sequencing of the peptides through MS/MS analysis, and sequence coverage of the fragment of AAG. The AAG fragment (SEQ ID NO: 1) is:

MALSWVLTVLSLLPLLEAQIPLCANLVPVPITNATLDQITGKWFYIASAF
RNEEYNKSVQEIQATFFYFTPNKTEDTIFLREYQTRQDQCIYNTTYLNVQ
RENGTISRYVGGQEHFAHLLILRDTKTYMLAFDVNDEKNWGLSVYADKPE
TTKEQLGEFYEALDCLRIPKSDVVYTDWKKDKCEPLEKQHEKERKQEEGE
S

The peptides fragments of SEQ ID NO: 1 have the following sequences:

| | |
|---|---|
| KWFYIASAFRN | (SEQ ID NO:2) |
| YVGGQEHFAHLLILR | (SEQ ID NO:3) |
| KTYMLAFDVNDEKN | (SEQ ID NO:4) |
| KNWGLSVYADKPETTKE | (SEQ ID NO:5) |
| KEQLGEFYEALDCLRI | (SEQ ID NO:6) |
| SDVVYTDWKK | (SEQ ID NO:7) |

FIG. 3 shows scatter plot of AAG concentrations measured by HPLC method. The serum AAG of 65 patients with HCC (37 patients with low AFP concentrations (≦200 ng/mL) and 28 patients with high AFP concentrations (>200 ng/mL)) and 54 non-cancer patients with chronic liver were shown. The dashed line indicates the cut-off point at 680 μg/mL. The dot lines represent the mean value with standard error mean. The mean value (±SEM) of AAG concentrations was higher in the patients with HCC (1129.82±83.45) μg/mL), especially HCC with low AFP (1245.86±121.17) μg/mL), than in the patients with chronic liver disease (409,89±22.90) μg/mL, (p≦0.0005).

FIG. 4 shows receiver operation characteristics (ROC) curve of the serum AFP, AAG, and the combination diagnose comprising logio serum AAG and logio serum AFP in the independent validation set. The clinical values were assessed by differentiating 65 HCC samples from 54 CLD samples.

### DESCRIPTION OF THE PREFERRED EMBODIMENTS

Before describing the present invention in detail, it is to be understood that this invention is not limited to particularly exemplified techniques, serum, media or methods and may, of course, vary. It is also to be understood that the terminology used herein is for the purpose of describing particular embodiments of the invention only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

The practice of the present invention will employ, unless otherwise indicated, conventional techniques of cell biology, cell culture, molecular biology, recombinant DNA, and immunology, which are within the skill of the art. Such techniques are described in the literature. See, for example, Sambrook et al, Molecular Cloning: A Laboratory Manual, 3^{rd} Ed., Cold Spring Harbor Laboratory Press, 2001); DNA Cloning, Volumes I and II (Glover ed., 1985); Oligonucleotide Synthesis (Gait, ed., 1984); Mullis et al. U.S. Pat. No. 4,683,195; Nucleic Acid Hybridization (Hames & Higgins eds. 1984); Transcription And Translation (Hames & Higgins eds. 1984); Meth. Enzymol., vols. 154 and 155 (Wu et al. eds.), Mayer and Walker (eds) Immunochemical Methods In Cell And Molecular Biology, Academic Press, London, 1987); Weir & Blackwell, eds., Handbook Of Experimental Immunology, Vols I-IV. 1986).

As used herein and in the appended claims, the singular forms “a,” “an,” and “the” include plural reference unless the context clearly dictates otherwise. Thus, for example, a reference to “a sample” includes a plurality of such samples, and a reference to “an antibody” is a reference to one or more antibodies, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

In its broadest aspect the present invention provides diagnostic markers for hepatocellular carcinoma (HCC). The term “marker” or “markers” as used herein refers to the markers for HCC described herein (which will also be referred to as the HCC markers). The HCC markers are AAG or orosomucoid (ORM) and isoforms thereof and AFP or glycoforms thereof, which are present in biological samples such as serum.

Alpha-1-acid glycoprotein (AAG) is a member of the lipocalins, a family that shares at least two structurally conserved sequence motifs (Flower, 1996, Biochem. J. 318:1-14). AAG is synthesized predominantly in the liver as a single polypeptide of 41-43 kDa, made up of 183 amino acids, with a hydrophobic prosthetic group, and a high content of sialic acid (Yuasa et al., 1997, Hum. Genet. 99:393-8). The biological functions of AAG are poorly understood; it is an acute phase protein, and its plasma level increase as a response to inflammation is triggered by cytokines (Bruno et al., 1998, J. Clin. Oncol. 16:187-96; Fournier et al., 2000, Biochim. Biophys. Acta; 1482:157-71). As a consequence, AAG levels vary in many physiological states (age and pregnancy) and pathological conditions such as liver cirrhosis, renal disease, and cancer (Kremer et al., 1988, Pharmacol. Rev. 40:1-47; Blain et al., 1985, Br. J. Clin. Pharmacol. 20:500-502; Duche et al., 2000, Clin. Biochem. 33:197-202.). Significant elevations of AAG have been found in patients with active lung and gastrointestinal cancers compared with patients with inactive disease. Moreover, in patients with colorectal cancer treated with 5-fluorouracil, AAG correlates with a response to therapy, with lower AAG levels seen in responding patients and higher AAG levels found in patients with progressive disease (Harshman et al., 1974, Cancer 34:291-9; Ganz et al., 1983, J. Natl. Cancer Inst. 71:25-30).

Alpha-fetoprotein (AFP) is a serum protein normally found at significant levels only in fetal blood. In adult blood, increased AFP levels are associated with liver regeneration and certain carcinomas. Accordingly, the term “alpha-fetoprotein” as used herein means a polypeptide having substantially the same amino acid sequence as the protein encoded by the AFP gene. See for example, T. Morinaga et al., 1983, Proc. Natl. Acad. Sci. USA 80:4604-8. Variants of AFP are also included in the term “alpha-fetoprotein,” as such variants are well known, for example, see U.S. Pat. No. 7,332,589 (Kubota et al., 2008) incorporated herein by reference in its entirety. If required, expression and purification of alpha-fetoprotein can be undertaken by the methods disclosed in U.S. Pat. No. 6,331,611.

It is known that AFP has a number of glycoforms (P, S, E, B, D). See for example a publication from the group of Taketa (E. Ichikawa et al., 2006, Electrophoresis 27:3480-7. Total AFP can be separated into three different glycoforms, AFP-L1, AFP-L2, and AFP-L3 depending upon their differential affinity to the lectin Lens culinaris agglutinin (LCA). AFP-L1 is the non-LCA-bound fraction, which constitutes the major glycoform of AFP in serum of chronic hepatitis and liver cirrhosis. AFP-L3 is the LCA-bound fraction of AFP. It has been reported that malignant liver cells produce AFP-L3, even when HCC is at its early stages, and especially when the tumor mass is supplied by the hepatic artery. Clinical research has determined that AFP-L3 is a highly specific marker for HCC.

In all cases, the methods of the invention require a biological sample to be obtained. “Biological samples” useful in the methods of the invention include, but are not limited to, a tissue, or fluid such as bone marrow, plasma, serum, spinal fluid, lymph fluid, the sections of the respiratory, intestinal, or genitourinary tracts, tears, saliva, milk, whole blood, tumors and organs.

In some embodiments, the biological sample is a body fluid. The term “body fluid” includes, for example, blood, blood serum, blood plasma, saliva, urine, bone marrow fluid, cerebrospinal fluid, synovial fluid, lymphatic fluid, amniotic fluid, nipple aspiration fluid and the like. Preferred body fluids for analysis are those that are conveniently obtained from patients, particularly preferred body fluids include blood serum and blood plasma.

The biological sample can be obtained from any mammalian subject suspected of having HCC. Thus, a “mammalian subject” suspected of having HCC can be any mammal including humans or mammals of economical importance and/or social importance to humans, for instance, carnivores other than humans (such as cats and dogs), swine (pigs, hogs, and wild boars), ruminants (such as cattle, oxen, sheep, giraffes, deer, goats, bison, and camels) and horses.

On presentation, for example, when a human subject is suspected of having a liver disease which might be HCC, a biological sample is taken by standard procedure. In some embodiments, blood is collected by any method or process known in the art. For example, use of a 21 gauge needle, 9 ml EDTA Vacuette™ tubes (catalogue No. NC9897284) and a syringe containing 0.225 ml of 10% neutral buffered solution containing formaldehyde (4% w/v). The methods or processes of collecting blood samples may also include other steps. For instance, blood collection devices may be modified to decrease cell lysis due to sheer forces in the collection needle, syringe or tubes used. For instance, needles of large gauge may be employed to reduce cell sheering or vacutainer tubes may be modified to reduce the velocity of blood flow. Any method may be used to isolate plasma from the cell components of blood after collection. The blood is best stored at 4° C. until processed.

Once taken, the biological sample may be analyzed directly, or may be treated prior to testing by, for example, concentration or pH adjustment. In some embodiments, the biological sample is serum obtained from blood taken from patients suspected of having CLD, HCC or at greater risk of developing HCC.

The “measuring,” “detecting,” or “quantitation” of a HCC marker of the present invention can be accomplished by any appropriate method including an immunological assay or a molecular biological assay. When the HCC “marker” is AAG or AFP polypeptide, the above method includes, for example, an immunological assay such as Enzyme Linked Immuno Sorbent Assay (ELISA), Radio Immuno Assay (RIA), fluorescent antibody technique, SDS-PAGE, Western blot or an immunohistochemical or other staining method.

When the HCC marker is an AAG or AFP polynucleotide such as mRNA, the assay includes a molecular biological assay, for example, Northern blot, Dot blot or reverse transcriptase polymerase chain reaction (RT-PCR). Messenger RNA can be measured, detected or quantitated by using an AAG or AFP polynucleotide or fragment thereof as a probe or primer.

In some embodiments, the “measuring,” “detecting,” or “quantitation” is by SDS-PAGE using a 12% polyacrylamide gel in accordance with Laemmli, Nature, 1970, 227:680-5. Once the SDS-PAGE gels have run for the required time they are stained with a commercial dye such as 0.25% Coomassie Brilliant Blue R250 (CBB) dissolved in 50% methanol-10% acetic acid to reveal the polypeptide bands. The presence or absence of the AAG or AFP polypeptides (HCC markers) of the present invention can be determined readily by assessing their size against a known standard.

Alternatively, in another preferred embodiment, the “measuring,” “detecting,” or “quantitation” of the HCC markers of the present invention is by Western Blot or ELISA. As appreciated by those skilled in the art, both of these techniques require the use of an antibody directed to the HCC polypeptides of the present invention. By using an antibody against the HCC markers or fragments thereof, the HCC markers can be measured, detected or quantitated.

Both monoclonal and polyclonal antibodies that bind to one of HCC markers of the present invention are useful in the methods and kits of the present invention. The antibodies can be prepared by methods known in the art.

To prepare polyclonal antibody, typically full length AAG or AFP polypeptide or a part thereof or a polypeptide which includes a part of the AAG or AFP polypeptide are given as an immunogen/antigen to a mammal. The polypeptide itself and a carrier, for example, a carrier combined with bovine serum albumin (BSA), keyhole limpet hemocyanin (KLH) or bovine thyroglobulin (BTG) can be used as an immunogen. To enhance immune responses to antigens, for example, complete Freund adjuvants (CFA) and incomplete Freund adjuvants (IFA) can be given. A mouse, a rat, a rabbit, a goat or a hamster can immunized. Well known methods for producing polyclonal antibodies can be found in Harlow, E. and Lane, D. Antibodies: A Laboratory Manual, Cold Spring Harbor Laboratory Press, Cold Spring Harbor, N.Y., 1989); Harlow, E. et al., Using Antibodies: A Laboratory Manual—Portable Protocol No. 1, Cold Spring Harbor Laboratory Press, Cold Spring Harbor, N.Y., 1998). Briefly, after the first immunization, a mammal is immunized with an appropriate antigen 3 to 10 times at 1 to 2-week intervals.

A preferred dosage of antigen is 50 to 100 μg at one time per an animal. When peptide antigens are used, peptides are preferably covalently bonded to appropriate carriers. Peptide antigens can be synthesized by methods of genetic engineering or peptide synthesis. Three to seven days after immunization, blood is collected and the responsiveness antibodies in the serum against the antigens can be measured by ELISA. See for example, Igaku-Shoin Ltd. (1976); Harlow & Lane, supra; Harlow et al., supra. Blood is then periodically collected from the immunized mammal until the immunized mammal shows a sufficient antibody titer, and then polyclonal antibodies can be prepared from the serum.

Separation and purification of polyclonal antibodies can be accomplished by chromatography such as a centrifugal separation, salting-out with ammonium sulfate, precipitation with caprylic acid, DEAE-sepharose column, anion exchange column, protein A column or G-column or a gel filter column (see, for example, Harlow & Lane, supra).

Once the mammal used to produce polyclonal antibodies has reached an appropriate titer they can also be used to prepare monoclonal antibodies (mAbs) against the AAG or AFP polypeptides of the present invention. In this procedure spleens or lymph nodes are extracted from the mammal and used to produce hybridoma by fusing an antibody-producing cell from the spleen or lymph node with a myeloma cell. As for the myeloma cell, cells established from a mouse or a rat can be used. Cell fusion can be done according to already known methods, for example, see Kohler and Milstein (1975) Nature 256:495-7).

The AAG or AFP polypeptides, parts thereof or polypeptides including the AAG or AFP polypeptides or parts thereof are injected into a rat. Three to seven days after the rat has shown a sufficient antibody titer, the rat is immunized with the antigen for the last time, and its spleen is extracted as antibody producing cells. The spleen is cut into pieces in MEM medium (Nissui Pharmaceutical Co. Ltd.) and the dissociated cells are precipitated by centrifugation at 1,200 rpm for 5 minutes. Splenocytes are separated by treating the precipitant with Tris-ammonium chloride buffer (pH 7.65) for 1 to 2 minutes to remove red blood cells. The splenocytes are washed with MEM medium 3 times and are used as antibody producing cells.

In order to establish a cell line myeloma cells are isolated from a mouse or rat. Appropriate myeloma cells can be isolated from the following strains: BALB/c (8-azaguanine resistance mouse), P3-X63Ag8-U1 (described as P3-U1) (Curr Topics Microbiol Immunol 81:1 (1978), SP2/0-Ag14 (described as SP-2) (Nature, 276:269 (1978)), P3-X63-Ag8653 (described as 653) (J. Immunol. 123:1548 (1979)) or P3-X63-Ag8 (described as X63) (Kohler & Milstein, supra). These cell strains are subcultured in a 8-azaguanine medium (a normal medium including 15 μg/ml 8-azaguanine (RPMI-1640 medium including 1.5 mM glutamine, 5×10⁻⁵ M 2-mercaptoethanol, 10 μg/ml gentamycin and 10% FCS made by CSL)) and cultured in a normal medium for 3 to 4 days before cell fusion. 2×10⁷ or more cells are prepared for cell fusion.

Hybridoma cells and myeloma cells are then mixed and washed with MEM medium or PBS (per 1 L; 1.83 g sodium phosphate dibasic, 0.21 g monobasic potassium phosphate, 7.65 g NaCl, pH7.2) and mixed as the number of antibody producing cells is 5 to 10 times larger than that of the myeloma cells. After a centrifugal separation at 1,200 rpm for 5 minutes, a precipitant is obtained. The precipitated cells are resuspended in 0.2 to 1 ml of polyethylene glycol solution (2 g polyethylene glycol-1000 (PEG-1000), 2 ml MEM medium, 0.7 ml dimethyl sulfoxide (DMSO)) per 10⁸ antibody producing cells is added to the cells with stirring at 37° C. 1 to 2 ml of MEM medium is then added several times every 1 to 2 minutes. The solution is prepared with MEM medium to 50 ml in total. After a centrifugal separation at 900 rpm for 5 minutes, a precipitant is obtained. 100 ml of HAT medium (normal medium including 10⁻⁴ M hypoxanthine, 1.5×10⁻⁵ M thymidine and 4×10⁻⁷ M aminopterin) is added to a precipitant and the precipitant is slowly resuspended. The suspension is poured into the 96-well culture plate at 100□1 per well and cultured at 37° C. in the presence of 5% CO₂ for 7 to 14 days.

By the methods described in Harlow & Lane, supra, and Harlow et al., supra, hybridomas producing antibodies specifically reacting with AAG or AFP polypeptides are selected.

Methods of detecting the HCC markers or parts thereof using the antibodies described above can involve but are not limited to direct or indirect bonded enzymes, fluorescent substances, radioisotopes or latexes. The assay method, for example, can be ELISA or a chemiluminescence method detecting enzyme activities such as horseradish peroxidase or alkaline phosphatase, FITC method detecting fluorescent tags such as luminol or GFP (Green Fluorescence Protein), RIA method detecting radioisotope tags such as ¹²⁵I or a latex agglutination method detecting binding with latex. The assay can also be, for example, Western blot or immune structure dyeing. Furthermore, the AAG or AFP polypeptides or parts thereof can be quantitated by the assay.

The antibodies used in the immunoassays can be immobilized to a solid phase carrier and the trapped polypeptides can be detected by using secondary antibodies with a reporter group or using reagents. Any substance, to which antibodies can attach and which is widely known to persons of ordinary skill in the art, can be used as a solid phase carrier. The substance includes, for example, a microtiter plate, a membrane such as a nitrocellulose membrane, bead, disk, glass, glass fiber, plastic material such as latex, polystyrene or polyvinyl chloride. Magnetic particles or fiber optical sensors (U.S. Pat. No. 5,359,681) can be used.

In this description, “solid phase” means immobilization by a physical method such as adsorption or a chemical binding by a covalent bond between an antibody and a functional group on a carrier. An antibody and a functional group on a carrier can be bonded directly or through a cross-linking agent. Immobilization by a physical method can be accomplished by appropriately diluted antibodies contacted with a carrier, preferably, a microtiter plate or a membrane in an appropriate buffer for an appropriate time. The contact time varies depending on the temperature, but it is typically between about 1 hour and 1 day. About 10 ng to 1 μg. preferably, about 100 to 200 ng of antibodies is added and immobilized on each well of a microtiter plate made of plastic such as polystyrene or polyvinyl chloride. Immobilization by a chemical method can be accomplished by a reaction of a carrier and functional groups of antibodies, for example, a reaction of a carrier and a two-functional reagent that reacts with both hydroxyl groups and amino groups and a carrier. For example, antibodies can be immobilized to a carrier having an appropriate polymer coat with a covalent bond by using benzoquinone or a condensation between aldehyde groups on a carrier and an amine or an active hydrogen on a combination partner.

A carrier-immobilized antibody is treated to inhibit physical adsorption of other polypeptides by a well-known method for a person having ordinary skill in the art with an appropriate blocking reagent, for example, cattle serum albumin or Tween 20 (Sigma-Aldrich). A carrier-immobilized antibody is reacted with a sample and polypeptides of the present invention and antibodies are combined. A biological sample can be appropriately diluted with an appropriate diluent, for example, phosphate buffered saline solution (PBS). A reaction time of a biological sample and antibodies should be enough to detect the presence of polypeptides of the present invention in a biological sample obtained from an subject suspected as having HCC, preferably, a time to achieve at least 95% of binding level compared to the level at which bound and not-bound polypeptides are equilibrated.

A time to reach equilibrium can be easily decided by measuring the binding level by the time. Substances other than bound polypeptides can be removed by washing a solid carrier with an appropriate buffer, for example, PBS (including 0.1% Tween 20). Labeled secondary antibodies are reacted with a solid carrier. The labels are preferably enzymes such as horseradish peroxidase, ground substances, supplemental elements, inhibitors, pigments, radioisotopes, coloring substances or fluorescent substances.

The binding between antibodies and labels can be accomplished by well-known methods. The secondary antibodies are reacted for a sufficient time to bind to complexes, which include immobilized antibodies and polypeptides of the present invention. An appropriate time can be easily decided by measuring binding level by the time. The non-binding secondary antibodies can be removed by washing a solid carrier with an appropriate buffer, for example, PBS (including 0.1% Tween 20).

The method of detecting or measuring labels of the secondary antibodies depends upon the kind of labels used. For example, when radioisotopes are used as labels, detection by a scintillation counter or an autoradiography can be used. When pigments, coloring substances or fluorescent substances are used as labels, detection by a spectrophotometer can be used. When enzymes are used as labels, substrates for the enzymes are added and reacted for a fixed time and the products are detected by a spectrophotometer. Labels and secondary antibodies can bind directly or indirectly by an avidin-biotin method. When they bind indirectly, one part of the avidin-biotin is bound to a secondary antibody and another is bound to a label. AAG or AFP polypeptides can also be detected by a flow through test or a strip test.

In a flow through test, a biological sample is added to a nitrocellulose membrane on which antibodies are immobilized, and when a sample passes through the membrane, polypeptides bind to the immobilized antibodies to form immune complexes. When a solution including labeled secondary antibodies passes through the membrane, it binds to the immune complexes. In a strip test, once a biological sample is added, the biological sample passes through a region including labeled antibodies, and polypeptides bind to labeled antibodies to form immune complexes.

When a biological sample passes through a region including a solid phase antibody, polypeptides bind to the immune complexes. The quantity of secondary antibodies detected in the region with immobilized antibodies shows the presence or absence of HCC.

An alternative to the “measuring,” “detecting,” or “quantitation” of the polypeptide of the present invention is the “detection,” “measuring” or “quantitation” of polynucleotides encoding the AAG or AFP polypeptides. The polynucleotides encoding the AAG or AFP polypeptides of the present invention can be used as the markers for HCC. The polynucleotide sequences encoding the AAG or AFP polypeptides of the present invention can be detected and measured using standard molecular-biologically techniques. Of particular interest are methods wherein an mRNA expressed by the genes encoding AAG or AFP are detected, measured or evaluated. Probes to detect mRNA are a nucleotide/deoxynucleotide probe that is complementary to and hybridizes with the mRNA and includes, but is not limited to, oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxigenin-labeled riboprobe (RNA probe) that is complementary to the mRNA encoding AAG or AFP is detected by binding the digoxigenin with an anti-digoxigenin secondary antibody and developed with nitro blue tetrazolium and 5-bromo-4-chloro-3-indoyl phosphate.

Whilst the probes may comprise double-stranded or single-stranded nucleic acid, single-stranded probes are preferred because they do not require melting prior to use in hybridizations. On the other hand, longer probes are also preferred because they can be used at higher hybridization stringency than shorter probes and may produce lower background hybridization than shorter probes.

Recommended pre-requisites for selecting oligonucleotide probes, particularly with respect to probes suitable for RT-PCR technology, are described in detail by Lockhart et al., 1996, Nature Biotech, 14, 1675-1680.

The nucleic acid probe may comprise a nucleotide sequence that is within the coding strand of the AAG or AFP genes. Such “sense” probes are useful for detecting RNA by amplification procedures, such as, for example, polymerase chain reaction (PCR), and more preferably, quantitative PCR or reverse transcription polymerase chain reaction (RT-PCR).

“Polymerase chain reaction,” or “PCR,” as used herein generally refers to a method for amplification of a desired nucleotide sequence in vitro, as described in U.S. Pat. No. 4,683,195. In general, the PCR method involves repeated cycles of primer extension synthesis in the presence of PCR reagents, using two oligonucleotide primers capable of hybridizing preferentially to a template nucleic acid. Typically, the primers used in the PCR method will be complementary to nucleotide sequences within the template at both ends of or flanking the nucleotide sequence to be amplified, although primers complementary to the nucleotide sequence to be amplified also may be used. See PCR Protocols, Academic Press, 1990, particularly Wang, et al., pp. 70-75 and Ochman, et al., pp. 219-227; Triglia et al., Nucl. Acids Res. 16:8186 (1988).

PCR may also be used to determine whether a specific sequence is present, by using a primer that will specifically bind to the desired sequence, where the presence of an amplification product is indicative that a specific binding complex was formed. Alternatively, the amplified sample can be fractionated by electrophoresis, e.g., capillary or gel electrophoresis, transferred to a suitable support, e.g. nitrocellulose, and then probed with a fragment of the template sequence. Detection of mRNA having the subject sequence is indicative of activation of the gene. “Oligonucleotides” or “oligonucleotide probes” are short-length, single- or double-stranded polydeoxynucleotides that are chemically synthesized by known methods (involving, for example, triester, phosphoramidite, or phosphonate chemistry), such as described by Engels, et al., 1989, Agnew. Chem. Int. Ed. Engl. 28:716-34. Typically they are then purified, for example, by polyacrylamide gel electrophoresis. Oligonucleotide probes of the invention are DNA molecules that are sufficiently complementary to regions of contiguous nucleic acid residues within the allergy-associated gene nucleic acid to hybridize thereto, preferably under high stringency conditions. Defining appropriate hybridization conditions is within the skill of the art. See e.g., Maniatis et al, in DNA CLONING, supra “Nucleic Acid Hybridization.” However, briefly, “stringent conditions” for hybridization or annealing of nucleic acid molecules are those that (1) employ low ionic strength and high temperature for washing, for example, 0.015M NaCl/0.0015M sodium citrate/0.1% sodium dodecyl sulfate (SDS) at 50° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5× SSC (0.75M NaCl, 0.075M sodium citrate), 50 mM sodium phosphate (pH 6.8), 0.1% sodium pyrophosphate, 5× Denhardt's solution, sonicated salmon sperm DNA (50 μg/mL), 0.1% SDS, and 10% dextran sulfate at 42° C., with washes at 42° C. in 0.2× SSC and 0.1% SDS.

Exemplary probes include oligomers that are at least about 15 nucleic acid residues long and that are selected from any 15 or more contiguous residues of DNA of the present invention. Preferably, oligomeric probes used in the practice of the present invention are at least about 20 nucleic acid residues long. The present invention also contemplates oligomeric probes that are 150 nucleic acid residues long or longer. Those of ordinary skill in the art realize that nucleic hybridization conditions for achieving the hybridization of a probe of a particular length to polynucleotides of the present invention can readily be determined. Such manipulations to achieve optimal hybridization conditions for probes of varying lengths are well known in the art. See, e.g., Sambrook et al., supra, incorporated herein by reference.

As used herein, the term “PCR reagents” refers to the chemicals, apart from the template nucleic acid sequence, needed to perform the PCR process. These chemicals generally consist of five classes of components: (i) an aqueous buffer, (ii) a water soluble magnesium salt, (iii) at least four deoxyribonucleotide triphosphates (dNTPs), (iv) oligonucleotide primers (normally two primers for each template sequence, the sequences defining the 5′ ends of the two complementary strands of the double-stranded template sequence), and (v) a polynucleotide polymerase, preferably a DNA polymerase, more preferably a thermostable DNA polymerase, i.e., a DNA polymerase which can tolerate temperatures between 90° C. and 100° C. for a total time of at least 10 minutes without losing more than about half its activity.

The four conventional dNTPs are thymidine triphosphate (dTTP), deoxyadenosine triphosphate (dATP), deoxycytidine triphosphate (dCTP), and deoxyguanosine triphosphate (dGTP). These conventional deoxyribonucleotide triphosphates may be supplemented or replaced by dNTPs containing base analogues which Watson-Crick base pair like the conventional four bases, e.g. deoxyuridine triphosphate (dUTP).

A detectable label may be included in an amplification reaction. Biotin-labeled nucleotides can be incorporated into DNA or RNA by such techniques as nick translation, chemical and enzymatic means, and the like. The biotinylated probes are detected after hybridization, using indicating means such as avidin/streptavidin, fluorescent-labelling agents, enzymes, colloidal gold conjugates, and the like. Nucleic acids may also be labeled with other fluorescent compounds, with immunodetectable fluorescent derivatives, with biotin analogues, and the like. Nucleic acids may also be labeled by means of attachment to a protein. Nucleic acids cross-linked to radioactive or fluorescent histone single-stranded binding protein may also be used. Those of ordinary skill in the art will recognize that there are other suitable methods for detecting oligomeric probes and other suitable detectable labels that are available for use in the practice of the present invention. Moreover, fluorescent residues can be incorporated into oligonucleotides during chemical synthesis. Preferably, oligomeric probes of the present invention are labeled to render them readily detectable. Detectable labels may be any species or moiety that may be detected either visually or with the aid of an instrument.

Suitable labels include but are not limited to fluorochromes, e.g. fluorescein isothiocyanate (FITC), rhodamine, Texas Red, phycoerythrin, allophycocyanin, 6-carboxyfluorexcein (6-FAM), 2′,7′-dimethoxy-4′,5′-dichloro-6-carboxyfluorescein (JOE), 6-carboxy-X-rhodamine(ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g., ³²P, ³⁵S, ³H, as well as others. Another group of fluorescent compounds are the naphthylamines, having an amino group in the alpha or beta position. Included among such naphthylamino compounds are 1-dimethylaminonaphthyl-5-sulfonate, 1-anilino-8-naphthalene sulfonate and 2-p-touidinyl-6-naphthalene sulfonate. Other dyes include 3-phenyl-7-isocyanatocoumarin, acridines, such as 9isothiocyanatoacridine acridine orange; N-(p-(2-benzoaxazolyl)phenyl)maleimide; benzoxadiazoles, stilbenes, pyrenes, and the like. Most preferably, the fluorescent compounds are selected from the group consisting of VIC, carboxy fluorescein (FAM), Lightcycler® 640 and Cy5.

The label may be a two stage system, where the amplified DNA is conjugated to biotin, haptens, or the like having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

RT-PCR is a form of PCR which can amplify a known mRNA sequence using a reverse transcriptase to convert the mRNA to cDNA prior to traditional PCR. In its simplest implementation, aliquots are removed from the PCR every couple of cycles beginning at a point where product is undetectable (typically about cycle 20) and extending through the entire exponential phase. Products are then resolved electrophoretically and quantitated by densitometry, fluorescence or phosphorimaging. Alternatively, a fluorescent signal can be used to report formation of PCR product as each cycle of the amplification proceeds, coupled with an automated PCR/fluorescent detection system (Heid et al., 1996, Genome Res.; 6:986-94). Suitable detection systems for real-time RT-PCR include SYBR® Green (Molecular Beacons), Scorpions® (Molecular Probes), and TaqMan® (Applied Biosystems).

The present invention also provides a method and kit for assaying the presence of AAG and AFP markers present in a biological sample taken from a mammalian subject suspected of having HCC. Early detection of the HCC can reduce the time for treatment and reduce the risk of developing clinically significant complications.

A simple point-of-care kit that uses principles similar to the widely-used serum testing kits, for the rapid detection of the HCC markers will allow the clinician to rapidly diagnose HCC, and to rapidly institute proven and effective therapeutic and preventive measures. The use of the kit can represent the standard of care for all patients who are at risk of developing HCC.

The methods and kits of the present invention can also provide a means for detecting or monitoring HCC including the change in status. It can be especially useful in detecting early stage HCC. Thus, the invention also provides a means for a clinician to monitor the progression of HCC (worsening, improving, or remaining the same) during and following treatment. Also, the methods and kits can monitor the potential progression of CLD into HCC. Typically, the clinician would establish a protocol of collecting and analyzing a quantity of biological sample from the patient at selected intervals. Typically the sample is obtained intermittently during a prescribed period. The period of time between intermittent sampling may be dictated by the condition of the subject, and can range from a sample each 24 hours to a sample taken days, weeks or even months apart.

Using the methods and techniques described herein, both a qualitative level of the HCC markers present in the biological sample can be analyzed and estimated, and a quantitative level of HCC markers present in the sample can be analyzed and measured. The clinician would select the qualitative method, the quantitative method, or both, depending upon the status of the patient. Typically, the quantity of sample to be collected is less than 3 ml, and more typically less than 1,000 μl . A typical sample can range from about 200 μl to about 2 ml.

In some embodiment, the term “comparing the quantified amount” refers to the comparison of the measured and/or determined amount of the AAG and AFP with a know standard or amount, namely, 680 μg/mL for AAG and 38 ng/ml for AFP, wherein any amount greater than these values is indicative of the presence of HCC.

In some embodiment, the term “comparing the quantified amount to a control sample”, refers to the comparison of the amounts of AAG and AFP in biological samples from mammals not suffering from hepatocellular carcinoma relative to the amounts of AAG and AFP found in the test biological samples i.e., those taken from subjects thought to have HCC.

The process of comparing a measured value of AAG and AFP from test subject samples and control samples can be carried out in any convenient manner appropriate to the type of measured value and control value for the AAG and AFP biomarkers at issue. For example, “measuring” can be performed using quantitative or qualitative measurement techniques as described supra, and the mode of comparing a measured value and a control value can vary depending on the measurement technology employed. In one example, the measured values used in the methods of the invention will most commonly be quantitative values (e.g., quantitative measurements of concentration, such as nanograms of AAG or AFP per milliliter of biological sample, or absolute amount). As with qualitative measurements, the comparison can be made by inspecting the numerical data, by inspecting representations of the data (e.g., inspecting graphical representations such as bar or line graphs).

Once an indication of HCC has been detected, and intervention and treatment of the condition has commenced, the clinician can employ the method and kit of the invention to monitor the progress of the treatment or intervention. Typically, one or more subsequent post-treatment samples will be taken and analyzed for the presence of HCC markers as the treatment of the HCC continues. The treatment is continued until the presence of the HCC markers in subsequent post-treatment samples are returned to a normal range. As the treatment and intervention ameliorate the condition, the expression of HCC markers (more specifically AAG and AFP), and their combined presence in the sample, will be correspondingly reduced. The degree of amelioration will be expressed by a correspondingly reduced level of the HCC markers detected in a sample.

A kit for use in the method typically comprises a media having affixed thereto one or more capture antibodies, whereby the sample is contacted with the media to expose the capture antibody to the HCC markers contained in the sample. The kit includes an acquiring means that can comprise an implement, such as a needle or vacutainer, having a surface comprising the media. The acquiring means can also comprise a container for accepting the sample, where the container has a serum-contacting surface that comprises the media. In another typical embodiment, the assay for detecting the complex of the HCC markers and the antibody can comprise an ELISA, and can be used to quantitate the amount of HCC markers in a sample. In an alternative embodiment, the acquiring means can comprise an implement comprising a cassette containing the media.

A method and kit of the present invention for detecting the HCC markers can be made by adapting the methods and kits known in the art for the rapid detection of other proteins and ligands in a biological sample. Examples of methods and kits that can be adapted to the present invention are described in U.S. Pat. No. 5,656,503, U.S. Pat. No. 6,500,627, U.S. Pat. No. 4,870,007, U.S. Pat. No. 5,273,743, and U.S. Pat. No. 4,632,901, all such references being hereby incorporated by reference.

A rapid one-step method of detecting the HCC markers of the present invention can reduce the time for detecting the development of HCC. A typical method can comprise the steps of: obtaining a sample from a mammal suspected of a predisposition to the development of HCC; mixing a portion of the sample with one or more detecting antibodies which specifically bind to one of the HCC markers, so as to initiate the binding of the detecting antibody to the HCC markers in the sample; contacting the mixture of sample and detecting antibodies with an immobilized capture antibody which specifically binds to the HCC markers, which capture antibody does not cross-react with the detecting antibodies, so as to bind the detecting antibodies to the HCC markers, and the HCC markers to the capture antibody, to form a detectable complex; removing unbound detecting antibody and any unbound sample from the complex; and detecting the detecting antibody of the complex. The detectable antibody can be labeled with a detectable marker, such as a radioactive label, enzyme, biological dye, magnetic bead, or biotin, as is well known in the art.

The invention will now be further described by reference only to the following non-limiting examples. It should be understood, however, that the examples following are illustrative, and should not be taken in any way as a restriction on the generality of the invention described herein. In particular, while the invention is described in detail in relation to the use of serum as the sample this does not preclude the use of other samples.

### EXAMPLES

### Patients

Serum samples from 119 patients were collected from the Hepatology Division at the Department Internal Medicine, Cipto Mangunkusumo Hospital, Indonesia. Sera were frozen immediately and stored at −80° C. before use. For each patient, clinical data, including age, sex, cause of diseases and AFP value, were collected. Diagnosis of HCC relied on the presence of a malignant liver nodule, as established on imaging techniques and by pathological analysis of liver biopsies. In total, there were 65 serum samples from patients with primary HCC at different clinical stages and with various AFP concentration (AFP≦200, n=37 and AFP>200, n=28). The control group consisted of 54 serum samples from patients with CLD only. At the moment of the first observation, informed consent to use clinical data and sera were obtained at the time of patient's diagnosis. Mean age±standard deviation (SD) for HCC and CLD patients were 54.25±13.57 and 46.15±13.99 years, respectively. Clinical characteristics of the patients are reported in Table 1.

### Serum AFP Determination

The qualitative measurement of serum AFP was performed using enzyme immunoassay method (Diagnostic System Laboratories, Webster, Tex. 77598 USA).

### Sample Preparation

Blood samples were centrifuged at 3800 rpm for ten minutes to separate the serum. All serum samples were stored at −80° C. in aliquots. Protein concentrations were determined with the Bradford method using Quick Start™ Bradford Dye Reagent 1X and Bovine Serum Albumin standard set (BioRad).

The level of AAG in each serum sample analysis was performed on an Agilent 1200 HPLC system. Two hundred microliters of crude serum were diluted with six hundred microliters of Buffer A (Agilent). Twenty microliters of the diluted serum were depleted on a Multiple Affinity Removal column (Agilent; 4.6 mm×50 mm) according to the manufacturer's instructions. The total volume of flow-through fraction (depleted serum collected at retention time between 2 and 6 min) approximately 1 ml was collected. The depleted serum was fractionated by column mRP-C18 (Agilent; 4.6×50 mm), operated at flow rate 0.75 ml/min and mobile phase comprised two solvents: A: water/0.1% trifluoro-acetic (TFA) and B: Acetonitrile/0.08% TFA. The AAG fraction was shown between retention times at 14.5-16 minute.

For protein identification purposes this method was repeated many times (1 mL crude serum) and all of fraction between retention time at 15-16 minute were collected and concentrated with a 10 kD Amicon Ultra Centrifugal Filter Devices (Millipore). The AAG fractions were lyophilized and used for protein identification analysis by using LC MS/MS.

### Measurement of Serum AAG Concentration

Quantification of serum AAG concentration was performed using ovalbumin as external standard (ESTD) and for calibration sample. Measurement of the standard was calculated based on peak area integration according to the Agilent Chemstation protocol. The isolated peak area was determined by the accumulation area above the baseline between peak start and stop (baseline to baseline peaks). The results from the serum sample between retention time at 14.5-16 minute (AAG peak) were compare with those of the calibration sample to calculate the amount of AAG.

### Polyacrylamide Gel Electrophoresis (SDS-PAGE)

SDS-PAGE was performed in a Mini-Protein III cell (Bio-Rad) using 12% gels with 0.1% SDS according to the manufacturer's instructions. Staining was performed with Coomassie™ Blue and used as prescribed by the manufacturer.

### Protein Identification by Mass Spectrometric (LC-MS/MS) Analysis

Coomassie-stained proteins were excised from the gels and then digested with trypsin (Promega Corporation, Madison, Wisc., USA), as previously described. Mass spectrometry analysis was performed at Research Center for Proteomic Analysis, Institute of Biochemistry and Cellular Biology, Chinese Academy of Sciences, Shanghai. LC MS/MS data of the tryptic peptide digest were searched via the Pro Found search engine to obtain the protein identity by undertaking the peptide mass fingerprinting approach. Tandem MS data were subjected to MS/MS ion search via the Mascot search engine to obtain the protein sequence of a particular peptide.

### Statistical Analysis

Comparison of serum AAG levels and clinical characteristics among the groups were analyzed using Mann Whitney U test (SPSS; SPSS In., Illinois). Receiver operating characteristics (ROC) curves were constructed by calculating the sensitivities and specificities of a biomarker or the diagnostic score of a logistic regression model at different cutoff points for differentiating HCC cases from chronic liver cases. The area under the ROC curves (AUC) can be statistically interpreted as the probability of the test to correctly distinguish the patients with HCC from CLD. An area of “1.0” represents a perfect test; an area of “0.5” represents a worthless test.

### Results

### Patients' Clinical Characteristics

The clinical background of HCC and CLD patients in this study are shown in Table 1. The group of patients with HCC included 65 patients (30.7% female and 69.3% male; mean age 54.25 years, range, 23-81 years). All patients displayed HCC related to either hepatitis B virus infection (70.7%), hepatitis C virus infection (20%) or unknown cause (9.3%). The control group of CLD patients comprised 54 patients (42.5% female and 57.4 male %; mean age 46.15 year, range, 18-77 years). Patients displayed CLD related to either hepatitis B virus infection (44.4%) or hepatitis C virus infection (55.6%). There was significantly different in the mean age between HCC and CLD patients (p<0.05). The mean value of the AST measurement between HCC and CLD group was significantly different (p<0.001), but this was not the case for the mean ALT value p=0.373).

### Protein Identification

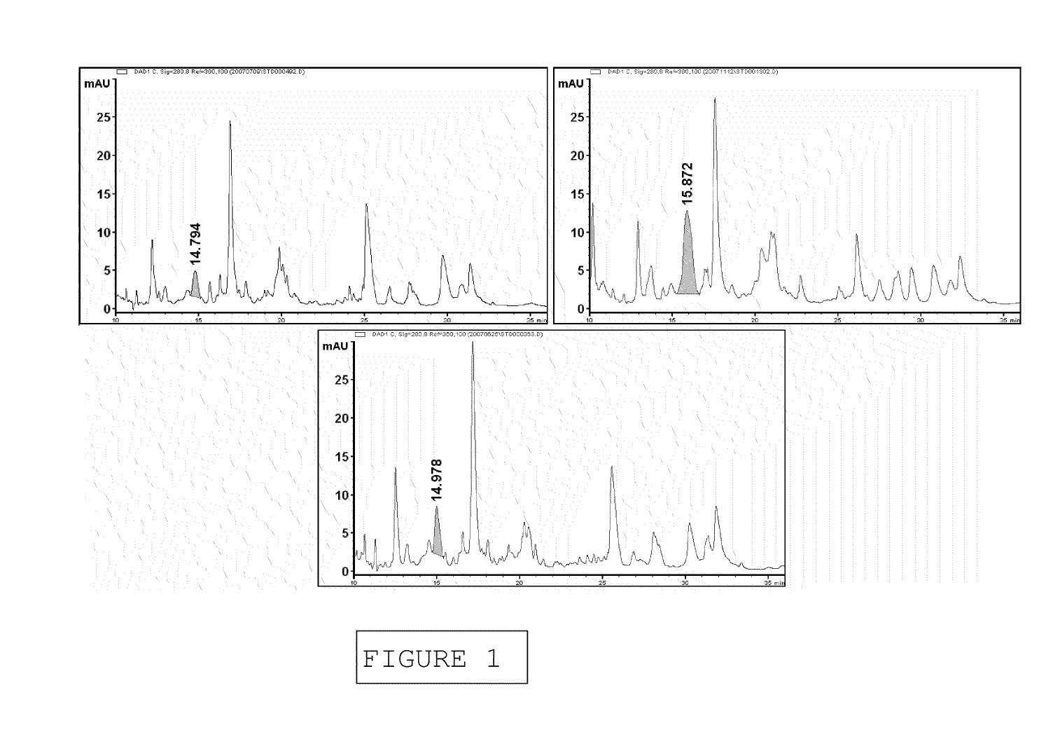

Depletion of high-abundance proteins is one way to enhance the capability of proteomic methods to detect suitable changes in the protein profile of human serum. After removal of seven highly abundant proteins from patient serum, a distinct peak at retention time 14.5-16 minute revealed high protein expression in staging of hepatocellular carcinoma HCC patients with low AFP (≦200 ng/ml, FIG. 1A) and high AFP concentration (≧200 ng/ml, FIG. 1B), but this peak was notably smaller in CLD patients (FIG. 1C).

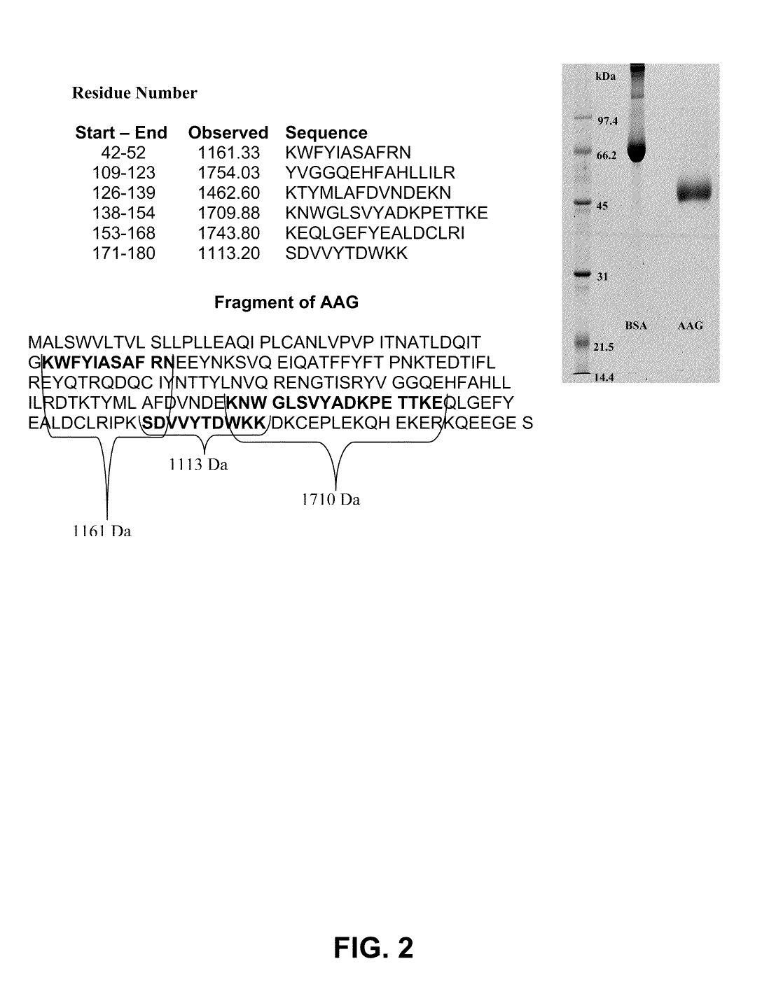

The identity of this protein was investigated using LC MS/MS. Peptide mass list derived from spectra with high S/N ration was submitted for database matching. We found that peptide fragments of this protein matched with those of AAG (FIG. 2).

### AFP and AAG Serum Concentration

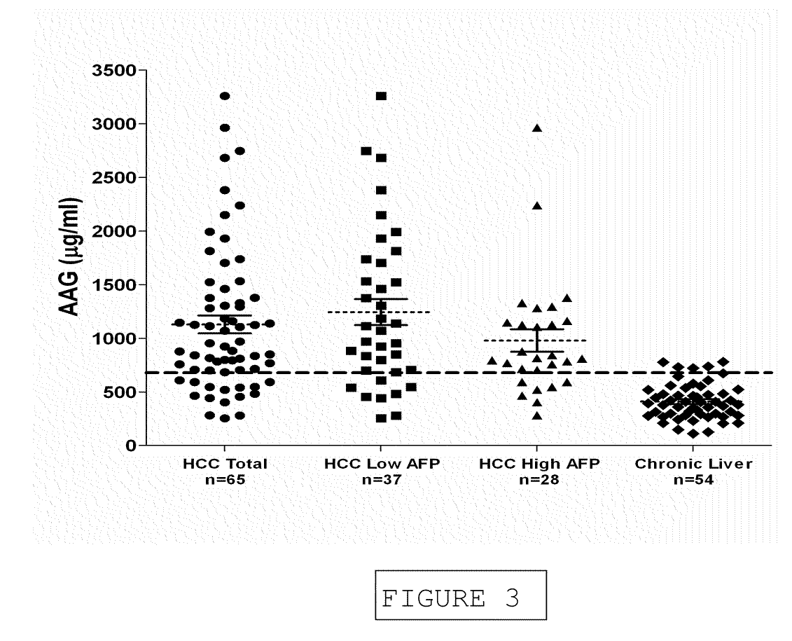

In the patients with HCC (n=65), the mean serum concentration [SD] of AFP was 14541.62 [32454.41] ng/mL. As expected, it was significantly higher than that of the CLD group (n=54) (16.39 [26.44] ng/mL, P<0.0005, Mann Whitney test). The mean serum concentration [SD] of total AAG in the HCC group was 1129.82[672.83] μg/mL, which was significantly higher than that of the CLD group (409.9 [22.9] μg/mL P<0.0005, Mann Whitney test, FIG. 3). The ratio of the mean values of HCC to CLD was 2.75. This confirms our earlier observation from chromatogram profile of CLD and HCC patients that serum AAG concentration is up-regulated in HCC patients.

When we differentiate between HCC patients with low and high serum AFP concentration, the mean serum concentration of total AAG in each group was 1245.86 (121.17) μg/ml and 979.14 (104.78) μg/ml respectively. The AAG serum concentration in both group was significantly higher than that of CLD patients (409.9 (22.9) μg/mL, P<0.0005, Mann Whitney test, FIG. 3)

### Comparison of Diagnostic Efficacy and Correlation Between AAG and AFP

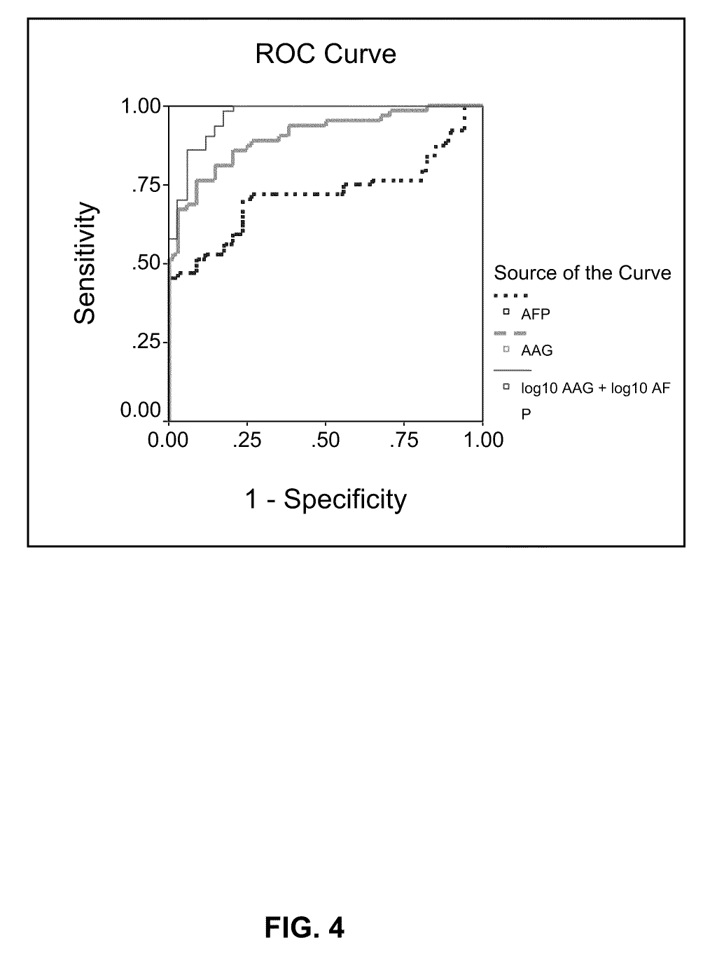

To evaluate the diagnostic value of serum AAG, ROC curve analysis was performed. The AUC of the ROC curve was 0.907 (95% CI: 0.855-0.960, P<0.0005, Table 2, FIG. 4), which was higher than that of serum AFP (AUC=0.750, 95% CI: 0.663-0.837, P<0.0005). The diagnostic sensitivity of AAG was 77% at 90% specificity with overall accuracy of 83%. The positive and negative likelihood ratios of serum AAG were 8.3 and 0.20, respectively. On the other hand, the diagnostic sensitivity of serum AFP was 52.30% at 90% specificity with overall accuracy of 70%. The positive and negative likelihood ratios of serum AFP were 5.6 and 0.50, respectively. This result shows that the diagnostic accuracy of serum AAG was higher than AFP for HCC under the current settings. This notion was further strengthened by the observation of 37 HCC patients who had serum AFP level less than 200 ng/mL. In stark contrast, the diagnostic efficacy of AAG among the same 37 patients was similar to the results from total 65 patients with HCC. Among 30 HCC patients who had AFP less than 20 ng/mL of serum AFP level, 25 patients (83.33%) showed higher serum AAG level than the presumptive cut-off level of 680 μg/mL.

### Diagnostic Value of Serum AAG in Combination of AFP

To evaluate the diagnostic value of AAG in combination with AFP, a multivariate logistic regression analysis was performed using the HCC and CLD cases. The logistic regression model was used to differentiate the HCC cases from the CLD cases in the independent validation set by calculating their diagnostic scores from logio AAG and logio AFP values. The AUC of the ROC curve of the diagnostic score was 0.943 (90% CI, 0.897-0.988, P<0.0005, Table 2). At 90% specificity, the sensitivity of this diagnostic model was 89%, which was much higher than the sensitivity obtained by using conventional marker AFP (52.31% at 90% specificity) or AAG alone (77% at 90% specificity). The overall accuracy of the combined diagnostic value was 90%. The positive and negative likelihood rations of the diagnostic model were 9.80 and 0.10, respectively.

This study clearly demonstrates that serum AAG concentration is increased in patients with HCC in comparison to CLD. We have shown that the diagnostic efficacy of AAG among the HCC patients with AFP level below 200 ng/mL was similar to the results from total HCC patients. Interestingly, 83.3% among HCC patients who had AFP level below diagnostic level (20 ng/mL) demonstrated higher AAG values. Since some patients even have low or negative AFP concentration in the terminal stage of their disease, these results allows us to propose that determining AAG levels in combination with AFP levels could be especially powerful in patients with HCC particularly with non diagnostic AFP levels.

The absence of correlation between AAG and AFP suggests that each marker may be related to a different aspect of HCC, therefore the use of them in combination could significantly improve diagnostic accuracy. This seems particularly important for those patients with normal levels of AFP whose disease might otherwise not have been recognized as HCC. By using serum AAG in combination with AFP, a diagnostic model for HCC detection with high sensitivity and specificity has been generated. In the independent validation set and at diagnostic specificity of 90%, the sensitivity for detection of HCC was 89.0%, which was much higher than the sensitivity (52.3%) when using AFP alone or AAG alone (77%).

The references cited above are all incorporated by reference herein in their entirety, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

What is claimed is:

1. A method of detecting hepatocellular carcinoma (HCC) in a mammalian subject, comprising:
(a) measuring the concentration of
(i) alpha-1-acid glycoprotein (AAG) or isoforms thereof, and
(ii) alpha-fetoprotein (AFP) or glycoforms thereof in a biological sample from a subject suspected of HCC; and
(b) comparing the concentration of said AAG or said isoforms and said AFP or said glycoforms in step (a) with control concentrations of AAG or isoforms thereof and AFP or glycoforms thereof that are present in biological samples from control subjects not suffering from hepatocellular carcinoma,
wherein an increased concentration of AAG or said isoforms and AFP or glycoforms measured in (a) compared to the control concentrations is indicative of the presence of HCC.

2. A method of detecting hepatocellular carcinoma in a mammal, said method comprising measuring the concentration of
(i) AAG or isoforms thereof, and
(ii) AFP or glycoforms thereof
in a biological sample from a subject suspected of HCC, wherein a concentration of AAG or said isoforms exceeding 680 μg/mL and a concentration of AFP or said glycoforms exceeding 38 ng/ml is indicative of the presence of HCC.

3. The method of claim 1 that further comprises the step of obtaining said sample from said subject.

4. The method of claim 2 that further comprises the step of obtaining said sample from said subject.

5. The method of claim 1, wherein the concentrations of AAG and AFP are measured.

6. The method of claim 2, wherein the concentration of AAG and AFP are measured.

7. The method of claim 1, wherein the subject is a human.

8. The method of claim 2, wherein the subject is a human.

9. The method of claim 1, wherein the biological sample is a tissue or a body fluid.

10. The method of claim 2, wherein the biological sample is a tissue or a body fluid.

11. The method of claim 9, wherein the body fluid is blood or a serum.

12. The method of claim 10, wherein the body fluid is blood or a serum.

13. The method of claim 1, further comprising observing a qualitative condition or measuring another quantitative indication of the subject's physical condition.

14. The method of claim 2, further comprising observing a qualitative condition or measuring another quantitative indication of the subject's physical condition.

15. A diagnostic kit for carrying out the method of claim 1, comprising reagents for detecting and/or measuring in a biological sample
(i) AAG or isoforms thereof, and
(ii) AFP or glycoforms thereof.

16. The kit of claim 15 wherein the reagents detect or measure AAG and AFP.

17. The kit of claim 15, wherein the reagents for measuring AAG and AFP are AAG-specific antibodies and AFP-specific antibodies, respectively.

18. The kit of claim 15, further comprising AAG and AFP each in a known quantity or concentration.

19. A competitive enzyme linked immunosorbent assay kit useful for detecting HCC in a mammalian subject, the kit comprising:
(i) a first antibody specific for AAG or for an isoform thereof, and
(ii) a second antibody specific for AFP or a glycoform thereof,
which antibodies bind to AAG and AFP, respectively, in a biological sample taken from said subject.

20. A point-of-care device for determining the presence and/or measuring the amount or concentration of AAG and AFP in a biological sample, which device comprises:
(a) a layer comprising a continuous porous medium deposited thereon;
(b) a fluid pathway defined by fluid barriers in the porous medium, at least a portion of the barriers defining a capillary channel, which fluid pathway comprises a constriction region immediately upstream of the capillary channel for constricting and directing fluid flow to the capillary channel;
(c) an analysis zone within the fluid pathway comprising a plurality of binding zones for binding and immobilizing (i) the AAG or isoforms thereof and (ii) the AFP or glycoforms thereof, each binding zone being defined by a concentration of a binding reagent immobilized to the medium; and
(d) an application zone at a first end of the fluid pathway and upstream of the analysis zone, for receiving a biological sample in which the AAG and AFP are to be measured.


SEQUENCE LISTING (7 sequences)

SEQ ID NO: 1 - protein, 201 aa, Artificial Sequence
  Fragment of Human Alpha-1-acid glycoprotein
MALSWVLTVLSLLPLLEAQIPLCANLVPVPITNATLDQITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 2 - protein, 11 aa, Artificial Sequence
  Fragment of Human Alpha-1-acid glycoprotein
KWFYIASAFRN

SEQ ID NO: 3 - protein, 15 aa, Artificial Sequence
  Fragment of Human Alpha-1-acid glycoprotein
YVGGQEHFAHLLILR

SEQ ID NO: 4 - protein, 14 aa, Artificial Sequence
  Fragment of Human Alpha-1-acid glycoprotein
KTYMLAFDVNDEKN

SEQ ID NO: 5 - protein, 17 aa, Artificial Sequence
  Fragment of Human Alpha-1-acid glycoprotein
KNWGLSVYADKPETTKE

SEQ ID NO: 6 - protein, 16 aa, Artificial Sequence
  Fragment of Human Alpha-1-acid glycoprotein
KEQLGEFYEALDCLRI

SEQ ID NO: 7 - protein, 10 aa, Artificial Sequence
  Fragment of Human Alpha-1-acid glycoprotein
SDVVYTDWKK